(19) United States
(12) Patent Application Publication
(10) Pub. No.: US 2025/0366905 A1
(43) Pub. Date: Dec. 4, 2025

(54) SLIP RING ASSEMBLIES FOR COUPLING ELECTROSURGICAL ENERGY TO ROBOTICALLY MANIPULATED SURGICAL INSTRUMENTS

(71) Applicant: KARL STORZ SE & Co. KG, Tuttlingen (DE)

(72) Inventors: Matthew Marchese, Cary, NC (US); Jonah Kadoko, Cary, NC (US)

(73) Assignee: KARL STORZ SE & Co. KG, Tuttlingen (DE)

(21) Appl. No.: 19/304,502

(22) Filed: Aug. 19, 2025

Related U.S. Application Data
(63) Continuation of application No. PCT/US2024/016582, filed on Feb. 20, 2024.
(60) Provisional application No. 63/485,908, filed on Feb. 19, 2023.

(51) Int. Cl. A61B 18/14; A61B 17/00; A61B 18/00; A61B 34/30

(52) U.S. Cl. CPC A61B 18/1482; A61B 34/30; A61B 2017/00486; A61B 2018/00077; A61B 2018/00083; A61B 2018/00178; A61B 2034/302

(57) ABSTRACT

An electrosurgical instrument having an elongate shaft and an adapter on the proximal end of the shaft is mountable to a robotic manipulator which can axially roll the instrument about its longitudinal axis. A connector on the adapter allows the adapter to be coupled to an electrosurgical unit so current can be conducted through the instrument to tissue in contact with the distal end of the instrument. The adapter includes a slip ring assembly that allows the connector to maintain its orientation relative to the electrosurgical instrument when the robotic manipulator is operated to axially roll the instrument and adapter.

### RELATED APPLICATIONS

This application is a continuation of International Application No. PCT/US2024/16582, filed Feb. 20, 2024, which claims the benefit of U.S. Provisional Application No. 63/485,908, filed Feb. 19, 2023.

### TECHNICAL FIELD OF THE INVENTION

The present invention relates generally to the field of surgical devices and systems, and more particularly to the field of surgical instruments for robot-assisted surgery.

### BACKGROUND

There are various types of surgical robotic systems on the market or under development. Some surgical robotic systems use a plurality of robotic manipulators or arms. Each manipulator carries a surgical instrument, or the camera used to capture images from within the body for display on a monitor. Typical configurations allow two or three instruments and the camera to be supported and manipulated by the system. Input to the system is generated based on input from a surgeon positioned at a surgeon console, typically using input devices such as input handles. The system responds to movement of a user input device by controlling the robotic manipulator that is associated with that input device to position, orient and actuate the surgical instrument positioned on that manipulator. The image captured by the camera is shown on a display at the surgeon console. The console may be located patient-side, within the sterile field, or outside of the sterile field.

Each robotic arm/manipulator includes a portion, typically at the terminal end of the arm, that is designed to support and operate a surgical device assembly. The surgical device assembly includes a surgical instrument having a shaft and a distal end effector on the shaft. The end effector is positionable within a patient. The end effector may be one of many different types that are used in surgery including, without limitation, end effectors having one or more of the following features: jaws that open and close, a section at the distal end of the shaft that bends or articulates in one or more degrees of freedom, a tip that rolls axially relative to the shaft, a shaft that rolls axially relative to the manipulator arm.

In many robotic surgical systems, including those using rigid shaft instruments, the surgical instruments are both robotically manipulated by the robotic manipulator arms disposed outside the patient's body, as well as electromechanically actuated within the patient's body. In many surgical systems, robotic manipulation using the arm pivots the instrument shaft relative to the incision site on the patient, and may also alter the insertion depth of the instrument into the patient and/or cause the instrument to roll about its longitudinal axis. Additionally, electromechanical actuation (or hydraulic/pneumatic actuation) may open and close jaws of the instrument, and/or actuate articulating or bending of the distal end of the instrument shaft, and/or roll the instrument's shaft or distal tip. Some systems may use only this latter form of instrument motion while holding the more proximal part of the instrument in a fixed position outside the body using a fixed support or inactive robotic manipulator.

A proximal housing is typically positioned on the proximal end of the instrument shaft. This housing functions as an adapter or interface between the surgical instrument and the robotic manipulator. The adapter may include passive actuation mechanisms that receive motion transferred from the active actuators in the robotic manipulator or other instrument support to drive functions of the instrument end effector. Such functions may include jaw open-close, shaft articulating or bending, or other functions. As noted above, the instrument actuators for driving the motion of the end effector, which respond to user input to cause actuation of the instrument's functions, are normally electromechanical motors, other types of motors or hydraulic/pneumatic actuators. They are often positioned in the terminal portion of the robotic manipulator. In some cases, they are positioned in the proximal housing of the surgical device assembly. In still other configurations, some are in the proximal housing while others are in the robotic manipulator. In the latter example, some functions of the end effector might be driven using one or more motors in the terminal portion of the manipulator while other motion might be driven using motors in the proximal housing. See, for example, US 2016/0058513, Surgical System with Sterile Wrappings, in which jaw open-close functions are initiated using electromechanical actuators in the robotic manipulator, and in which rotation or swivel functions of the instrument are initiated using electromechanical actuators housed in the proximal housing of the surgical device assembly.

Various systems have different types of mechanical interfaces between the adapter and the robotic manipulator. It is through these interfaces that motion generated by the instrument actuators within the robotic manipulator is communicated to one or more mechanical inputs of the adapter to control degrees of freedom of the instrument and, if applicable, its jaw open-close function. This motion may be communicated through a sterile drape positioned between the sterile adapter and the non-sterile manipulator arm. In some commercially available robotic systems, the motion is communication using rotary connections in which rotating disks on the manipulator transfer motion to rotating disks on an instrument adapter. See, for example, the configuration shown in U.S. Pat. No. 6,491,701. In others, such as the embodiment shown in U.S. Pat. No. 9,358,682, a transverse slider pin extends laterally from one side of the case mounted to the proximal end of the instrument. It is moveable to open and close jaws of the instrument (FIG. 18 of the patent). When the instrument is mounted to the manipulator arm, the slider pin is received by a corresponding component 430 (FIG. 19) in the manipulator arm. When it is necessary to open/close the instrument jaws, the component 430 is translated on a carriage by motors in the laparoscopic instrument actuator 400 of the manipulator arm. This advances the slider pin 314 to actuate the jaws.

Yet another adapter configuration is shown and described in commonly owned US Publication No. 2021/169595. In that configuration, the adapter of a surgical instrument includes a pair of planar faces on opposite sides of the adapter. Two longitudinally slidable drive inputs are exposed at each face, giving the adapter a total of four drive inputs. The instrument is engaged to a robotic manipulator by positioning the instrument adapter between two arms of an expandable instrument drive system, and then closing the arms to capture the adapter between the arms. This engages each drive input with a corresponding drive output on the instrument drive system. The use of four drive inputs in this configuration allows for both jaw open-close functions and articulation of the end effector in both pitch and yaw directions.

The instruments are exchangeable during the course of the procedure, allowing one instrument (with its corresponding adapter) to be removed from a manipulator and replaced with another instrument and its corresponding adapter.

In some systems, some of the surgical instruments may be removably connected to their respective adapters. This facilitates post-surgery cleaning and sterilization of the instruments and adapters, by allowing them to be separated for cleaning and sterile processing.

Electrosurgical instruments of various types are frequently used in performing surgery. Non-limiting examples include monopolar scissors and hooks, and bipolar graspers. Such instruments require the coupling of a line from an electrosurgical generator unit to the instruments. This application describes an arrangement by which the line may be coupled to the instrument without becoming tangled during movement of instrument by a robotic manipulator.

### BRIEF DESCRIPTION OF THE DRAWINGS

FIG. 1 is a perspective view of a robot-assisted surgical system on which the configurations described herein may be included

FIG. 2 is a perspective view of a robotic manipulator arm with the receiver/IDS and instrument assembly mounted to it.

FIG. 3 are a perspective view showing the IDS of FIG. 2 and the surgical instrument separated from the receiver.

FIG. 4 is an exploded perspective view of a slip ring assembly for a bipolar instrument.

FIG. 5A shows the slip rings of FIG. 4 with the conductive wires or cables attached to them.

FIG. 5B shows the slip rings of FIG. 5B positioned on the flange during the assembly of the slip ring assembly.

FIG. 5C is a partially assembled view showing the slip rings positioned on the flange, the brushes in contact with the slip rings, and the connector pins assembled with the brushes.

FIG. 5D is a partially assembled view showing a brush and the connector pins assembled with the center housing. The brush and its corresponding connector pin are shown shaded to make them more visible.

FIG. 5E is a cross-section through between the slip ring assembly and distal portion of the assembly.

FIG. 6 is an assembled view of the slip ring assembly of FIG. 4.

FIG. 7 is a perspective view of a bipolar surgical instrument which includes the slip ring assembly of FIGS. 4-6;

FIG. 8 is an exploded perspective view of a slip ring assembly for a monopolar instrument;

FIG. 9 is an assembled view of the slip ring assembly of FIG. 9;

FIG. 10 is a perspective view of a bipolar surgical instrument which includes the slip ring assembly of FIGS. 8 and 9.

### DETAILED DESCRIPTION

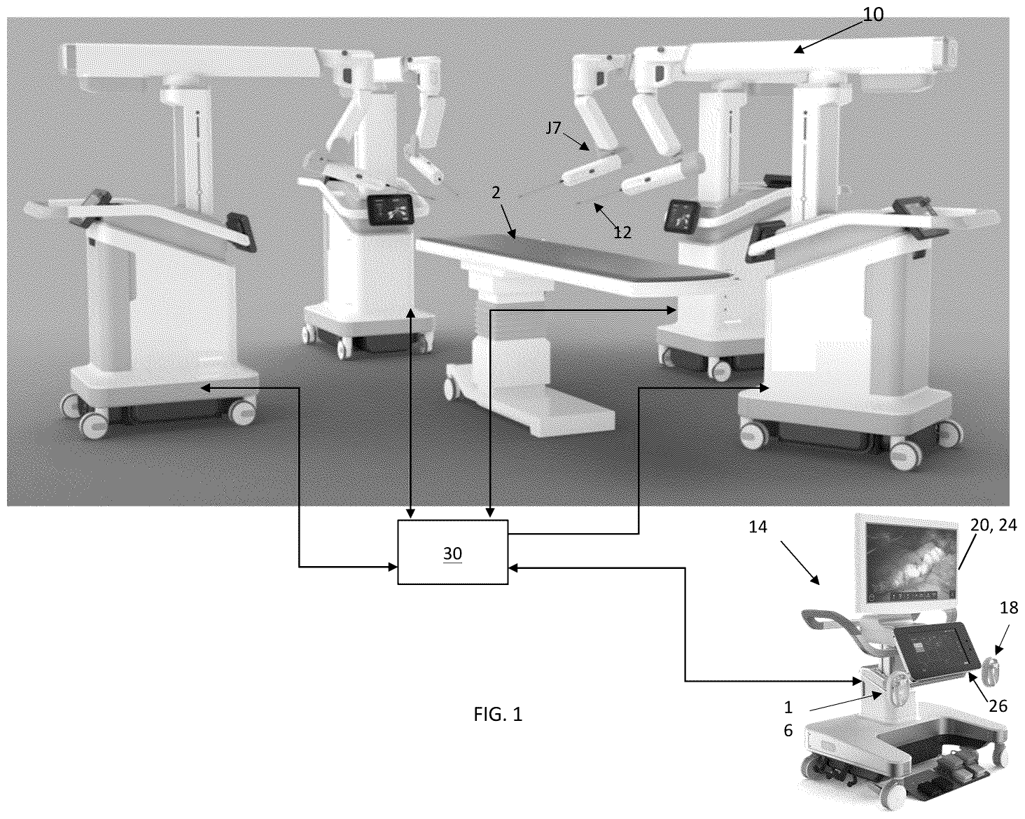

Although the concepts described herein may be used on a variety of robotic surgical systems, the embodiments will be described with reference to a system of the type shown in FIG. 1. In the illustrated system, robotic manipulators 10 are disposed adjacent to a patient bed 2. Each manipulator 10 is configured to maneuver a surgical instrument 12 which has a distal end effector positionable in a patient body cavity. FIG. 1 shows four robotic manipulators, although in other configurations, the number of manipulators may differ.

A surgeon console 14 has two input devices such as handles 16, 18. The input devices are configured to be manipulated by a user to generate signals that are used to command motion of the robotic manipulators in multiple degrees of freedom in order to maneuver the instrument end effectors within the body cavity. The input devices may be mounted to linkages, gimbals, etc. equipped with sensors that generate signals corresponding to positions or movement of the input devices in manners known to those skilled in the art. In other embodiments, the input devices may take the form of handles that are tracked using a tracking system, such as an optical tracking system or an electromagnetic tracking system, either alone or in combination with other sensors within the handles, such as IMUs etc.

In use, a user selectively assigns the two handles 16, 18 to two of the robotic manipulators 10, allowing surgeon control of two of the surgical instruments 12 at any given time. To control a third one of the instruments disposed at the working site, one of the two handles 16, 18 may be operatively disengaged from one of the initial two instruments and then operatively paired with the third instrument, or another form of input may control the third instrument as described in the next paragraph.

One of the instruments 12 is a camera that captures images of the operative field in the body cavity. The camera may be moved by its corresponding robotic manipulator using input from a variety of types of input devices, including, without limitation, one of the handles 16, 18, additional controls on the console, a foot pedal, an eye tracker 20, voice controller, etc. The console may also include a display or monitor 24 configured to display the images captured by the camera, and for optionally displaying system information, patient information, etc. An auxiliary display 26, which may be a touch screen display, can further facilitate interactions with the system.

The surgical system allows the operating room staff to remove and replace the surgical instrument 12 carried by a robotic manipulator 10, based on the surgical need. When an instrument exchange is necessary, surgical personnel remove an instrument from a manipulator arm and replace it with another.

As discussed, manipulation of the input devices 16, 18 results in signals that are processed by the system to generate instructions for commanding motion of the manipulators in order to move the instruments in multiple degrees of freedom and to, as appropriate, control operation of electromechanical actuators/motors that drive instrument functions such as articulation, bending, and/or actuation of the instrument end effectors. One or more control units 30 are operationally connected to the robotic arms and to the user interface. The control units receive user input that is generated as a result of movement of the input devices, and generates commands for the robotic arms to manipulate the surgical instruments so that the surgical instruments are positioned and oriented in accordance with the input provided by the user.

Sensors in the robotic manipulators determine the forces that are being applied to the patient by the robotic surgical tools during use. U.S. Pat. No. 9,855,662, entitled Force Estimation for a Minimally Invasive Robotic Surgery System, which is incorporated herein by reference, describes a method by which input from a 6 DOF force/torque sensor on the robotic manipulator is used to determine the RCM about which the surgical instrument should be pivoted, which corresponds to the location of the incision along the instrument shaft. Motion of the robotic manipulator is thus algorithmically controlled to constrain the motion such that the instrument pivots relative to the RCM. In the presently disclosed embodiments, a sensor of this type may be optionally positioned just proximal to the instrument drive system 104. It should be understood that use of the 6 DOF force/torque sensor to determine the RCM is desirable, but is not essential for practice of the present invention. Other methods for determining the location of the RCM, including those discussed in the Background, may be used in the presently-described manipulator arm in lieu of methods using the 6 DOF force/torque sensor.

Referring to FIGS. 3, positioned at the distal end of each manipulator arm is a receiver 104, which may also be referred to as an instrument drive assembly (IDS). A different surgical instrument 12 is removably mountable to each IDS. As best seen in FIG. 3, each instrument 12 includes an elongate shaft 106, which is preferably rigid but which may be flexible or partially flexible in alternative systems. An end effector 108 is positioned at the distal end of shaft 106, and a base assembly or adapter assembly 110 is at the proximal end.

Instrument and IDS configurations suitable for use with the disclosed inventions will next be described, but it should be understood that these are given by way of example only. The disclosed manipulator may be used with various configurations of instruments and instrument drive systems. More particularly, while the receiver/IDS described here is configured to drive pitch and jaw motion of an articulated surgical instrument, in alternative embodiments the receiver/IDS may have less functionality. In some alternative configurations, it may serve simply to receive an instrument and to drive jaw open/close operations. In other configurations, it may be configured, along with the instrument, to actuate a roll function of the instrument tip relative to the shaft of the instrument.

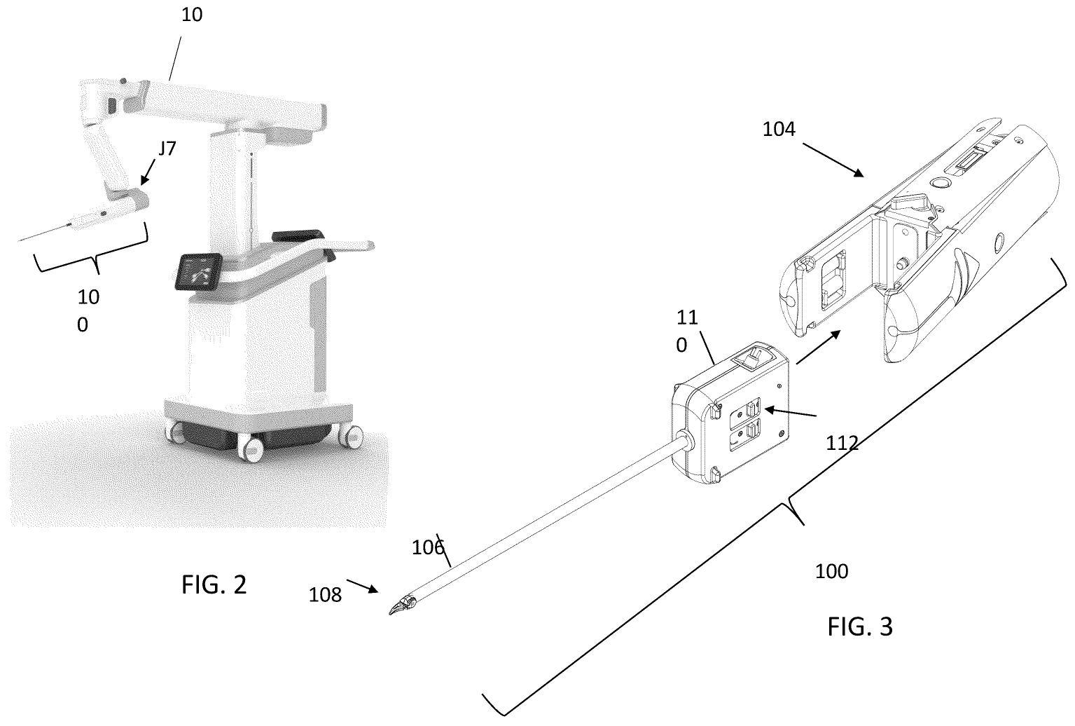

The instrument depicted in the drawings is the type described in Applicant's commonly-owned co-pending application published as US 2020/0375680, entitled Articulating Surgical Instrument, which is incorporated herein by reference. It makes use of four drive cables two of which terminate at one of the jaw members and the other two of which terminate at the other jaw member. This can be two cables looped at the end effector (so each of the two free ends of each cable loop is at the proximal end) or it can be four individual cables. As described in the co-pending application, the tension on the cables is varied in different combinations to effect pitch and yaw motion of the jaw members and jaw open-close functions. Other instruments useful with the system will have other numbers of cables, with the specific number dictated by the instrument functions, the degrees of freedom of the instrument and the specific configuration of the actuation components of the instrument. Note that in this description the terms “tendon,” “wire,” and “cable” are used broadly to encompass any type of tendon that can be used for the described purpose. The surgical instrument's drive cables extend from the end effector 108 through the shaft 106 (FIG. 2) and extend into the adapter assembly 110 where they are coupled to mechanical actuators. A more detailed description is given in Applicant's co-pending application published as US 2021/169595, which is incorporated herein by reference, but a general configuration of these actuators with respect to the adapter assembly will be provided here.

The adapter assembly 110 (which will also be referred to as the “adapter”) may include an enclosed or partially enclosed structure such as a housing or box, or it may be a frame or plate. The exemplary adapter 110 shown in the drawings includes mechanical input actuators 112 exposed to the exterior of the surgical instrument 102. In FIG. 3, two mechanical input actuators 112 are exposed at a first lateral face of the adapter 110. A second two mechanical input actuators 112 (not visible in FIG. 3) may be exposed at the second, opposite, lateral face of the adapter 110, preferably but optionally in a configuration identical or similar to the configuration shown in FIG. 3.

Each of the mechanical input actuators 112 is moveable relative to the adapter 110 between first and second positions. In the specific configuration shown in the drawings, the actuators are longitudinally moveable relative to the housing between a first (more distal) position and a second (more proximal) position such as that shown in FIG. 3. The direction of motion, however, is not required to be longitudinal and can extend in any direction.

In this configuration, the adapter thus has four drive inputs, one for each of the input actuators 112, exposed to its exterior. The illustrated adapter has two parallel planar faces, with two of these inputs positioned on each of the faces. While it may be preferred to include the inputs on opposite sides of the proximal body, other arrangements of inputs on multiple faces of the proximal body can instead be used. Each of these configurations advantageously arranges the drive inputs to maximize the distance between control inputs, minimizing stresses in the sterile drape that, in use, is positioned between the proximal body and the receiver 104. Co-pending US 2021/169595 includes further description of the adapter shown in FIG. 3.

The IDS 104 at the end of each manipulator 10 has an open position (shown in FIG. 3) in which it removably receives the adapter 110 of a corresponding instrument 12, to form an assembly 100. After the adapter 110 is placed within the IDS, the IDS is moved to the closed position shown in FIG. 2, capturing the adapter 110. In this position, the drive inputs 112 of the adapter can engage with corresponding drive outputs 114 of the IDS. As described in detail in co-pending US 2021/169595, user input at the input devices 16, 18 commanding jaw open-close, pitch or yaw articulation etc. of the instrument causes electromechanical actuators in the IDS to move the drive outputs 114. The motion of those drive outputs moves corresponding ones of the adapter's drive inputs 112, altering tension on the instrument's drive cables in a manner that causes the desired motion at the instrument's end effector.

### Slip Ring Assembly

Referring again to FIG. 2, the distalmost joint J7 of the manipulator arm is one configured to permit 360 degrees of axial roll, resulting in axial rotation of the IDS 104, adapter 110 and instrument 106 about the longitudinal axis extending between the distal and proximal ends of the instrument. The present inventors have developed a slip ring assembly that provides a means to connect bipolar or monopolar energy to an instrument adapter capable of rolling continuously. This invention allows the electrical connection to maintain a constant orientation with respect to the ground, preventing ESU cable from winding up around the IDS as the instrument rolls continuously.

The electrical contact is created using a C-shaped sheet metal arms/brushes which are spring loaded against a metal ring (as seen in the drawings).

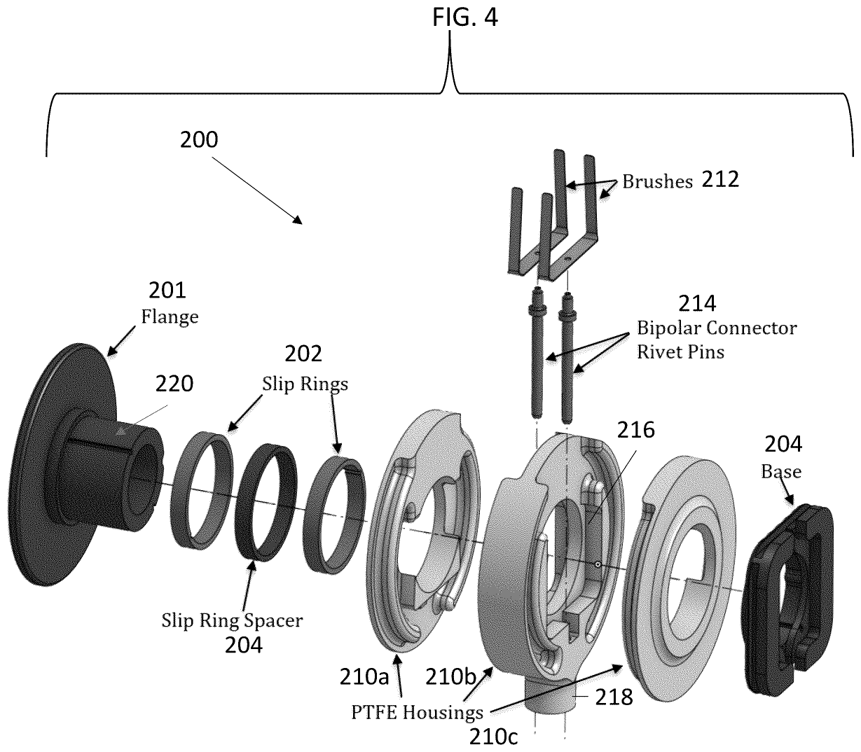

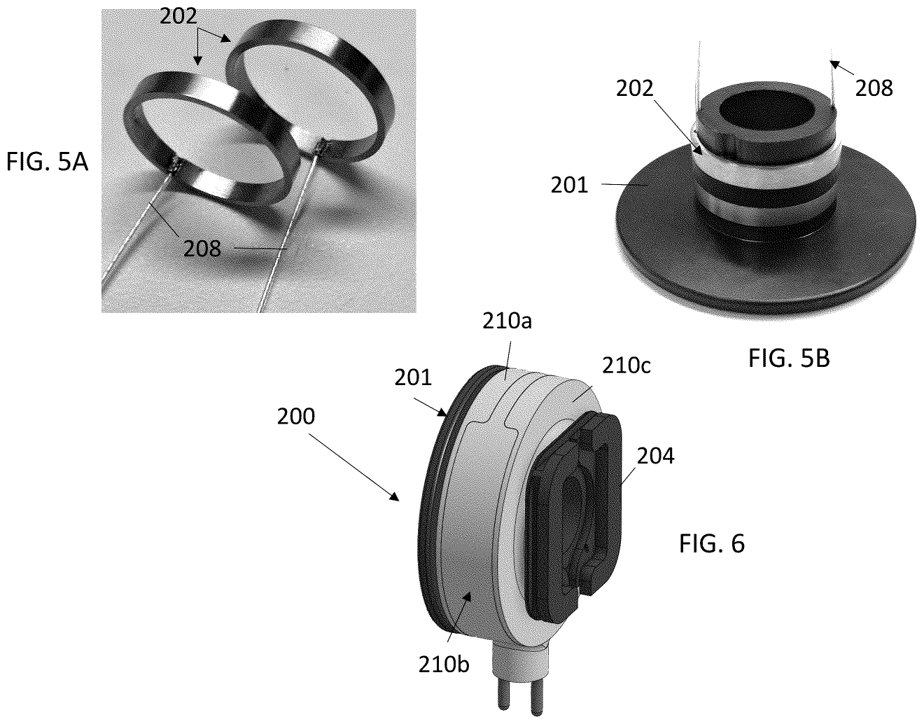

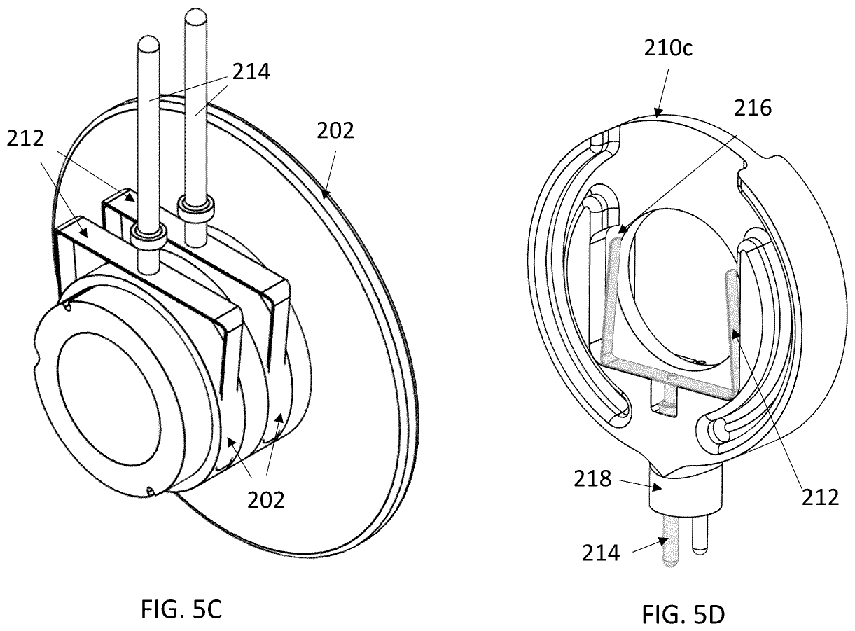

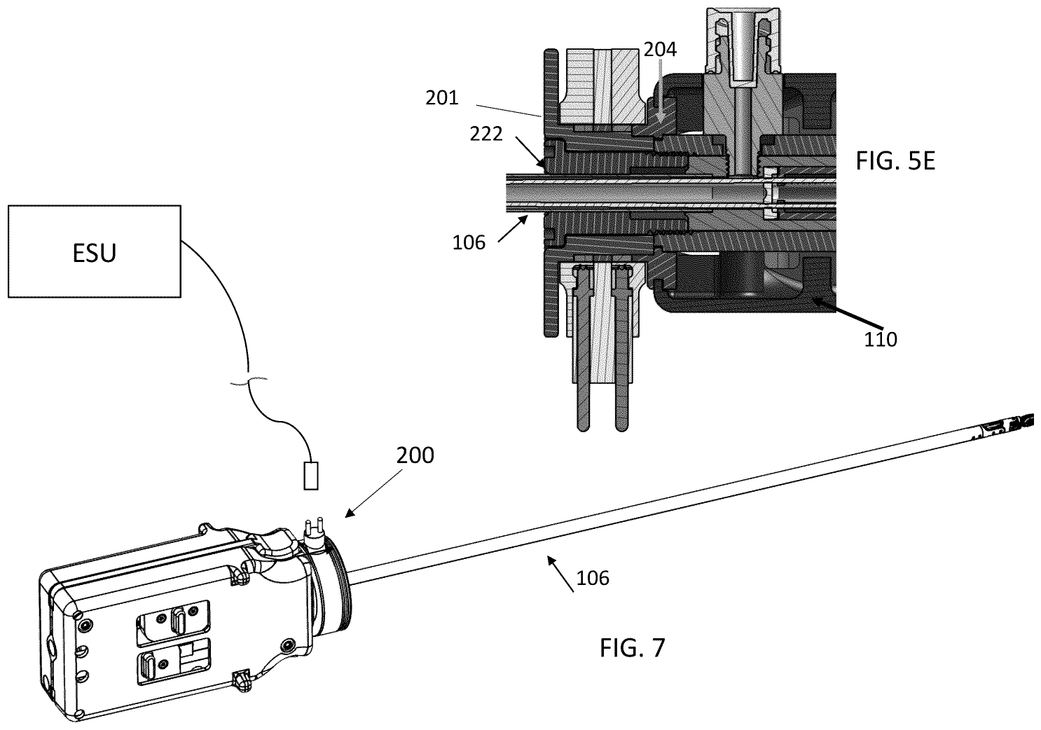

FIG. 4 shows an exploded view of a bipolar slip ring assembly 200. The outer pieces of the slip ring are a flange 201 and a base 204. These comprise the portion of the slip ring frame which is fixed to the adapter 110. These components may be fabricated out of PEEK for its mechanical and dielectric strength, biocompatibility, chemical compatibility, and ability to withstand a large number of autoclave cycles without mechanical degradation. Electrically conductive slip rings 206 are disposed around the shaft of the flange 201, and are spaced apart by an electrically non-conductive spacer. As shown in FIGS. 5A and 5B, conductive wires 208 are attached to the slip ring. These conductive wires extend to the jaws of the end effector of the surgical instrument in order to create a conductive pathway to the end effector. The wires are preferably made of stranded, silver plated copper with PFA or FEP insulation. The wires may crimped with ferrules which are welded to the slip rings. When the slip ring assembly is assembled, the distal slip ring wire is routed underneath the spacer ring and subsequently underneath the proximal slip ring through a groove 220 in the slip ring flange. The wire insulation prevents it from shorting to the proximal slip ring.

A housing formed by housing sections 210a, 210b, 210c are disposed between the flange and base. The drawings show three such housing sections, including a center housing section 210b with seats 216 formed on opposite faces of the center housing section (only one seat is visible in FIG. 4). Seats 216 are shaped to receive the rectangular C-shaped electrical brushes 212 while maintaining electrical separation between them using an intermediate wall section. The brushes 212 are are spring loaded against the slip rings 202, with the inner surface of each rectangular “C” contacts the outer surface of a corresponding one of the slip rings 206 as shown in FIG. 5C.

The end housing sections 210a and 210c mate with opposite faces of the housing section 210b, sandwiching each brush 212 between the center housing section 210b and the adjacent one of the end housing sections 210a, 210c. This electrically isolates each brush from other brush using the center housing section 210b, as shown. As best seen in FIG. 5D, within the housing section 210c, bipolar connector pins 214 are connected to the electrical brushes 212 and extend out through a connector 218 formed in the center housing section 210b, and thus create a conductive pathway from each connector pin to its corresponding brush, and thus to the corresponding slip ring and conductive wire (not shown in FIG. 5D, but scc FIG. 5A).

It will be noted that each of the housing sections 210a-c includes a central opening. When the assembly 200 is fully assembled as shown in FIG. 6, the shaft of the flange 201 extends through the slip rings 202, spacer 204, housing sections 210a-c and base 204. A compression plug may be extended through the assembly and attach, such as by a screw fitting, to between the flange 201 and base 204 hold the assembly together. The connector pins extend from the housing section 210b and can be removably connected with the cable for an electrosurgical surgical unit (ESU) as depicted in FIG. 7. Although the ESU places the connector pins at opposite polarity, the center housing 210b maintains electrical isolation between them. Housing sections 210a-b and the ESU cable connection 218 extending from the center housing sections are fabricated out of solid PTFE for its low friction coefficient, excellent dielectric strength, biocompatibility, chemical compatibility, and ability to withstand are large number of autoclave cycles without mechanical degradation.

The slip ring assembly is assembled with an adapter as shown in FIG. 7 and as described below. When the adapter is mounted to the IDS (FIGS. 2 and 3), a cable from an ESU is coupled to the connector extending from the housing to electrically couple the instrument to the ESU. The robotic arms 10 include a joint J7 (FIG. 1) that causes axial roll of the instrument about its longitudinal axis. When the ISU and instrument are rotated about the longitudinal axis of the instrument shaft due to motion of joint J7, the orientation of the connector 218 does not change. Instead, the flange 201 and base 204 rotate with the adapter and relative to the housing, while the housing 210a-210c maintains its orientation. The slip rings 202 and their associated wires 208 rotate with the flange 201, with the slip rings maintaining contact with the brushes retained in the housing.

The housing sections are held together with 2-part autoclavable epoxy. The base and flange are keyed together to prevent any rotation between the parts. Once fully assembled, the slip ring assembly is keyed onto the adapter's frame and covers, and the flange is locked against the frame with a compression nut 222 (FIG. 6E) that also retains the shaft assembly for extra support.

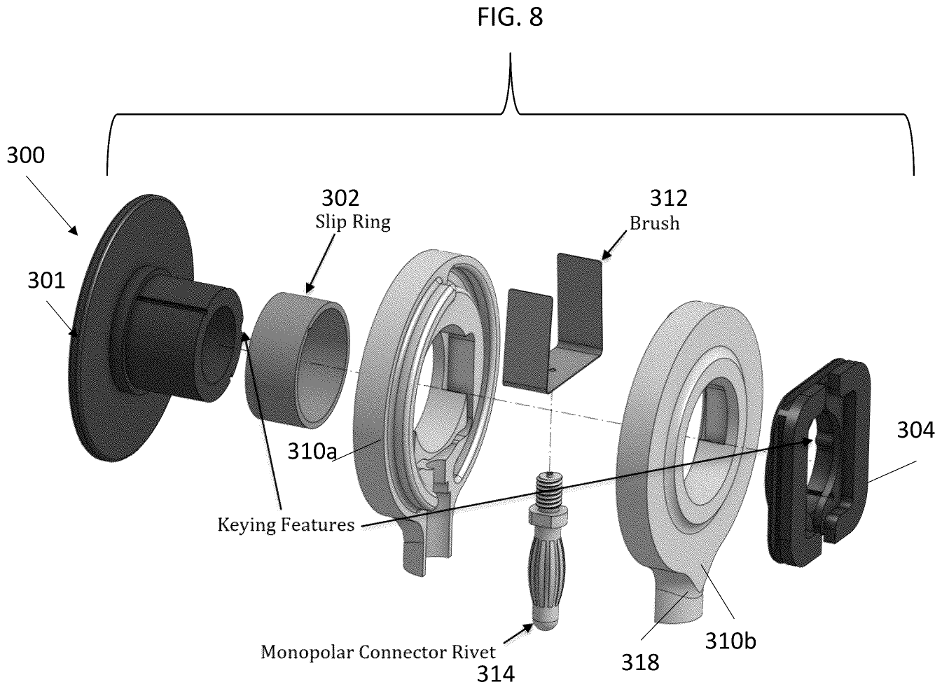

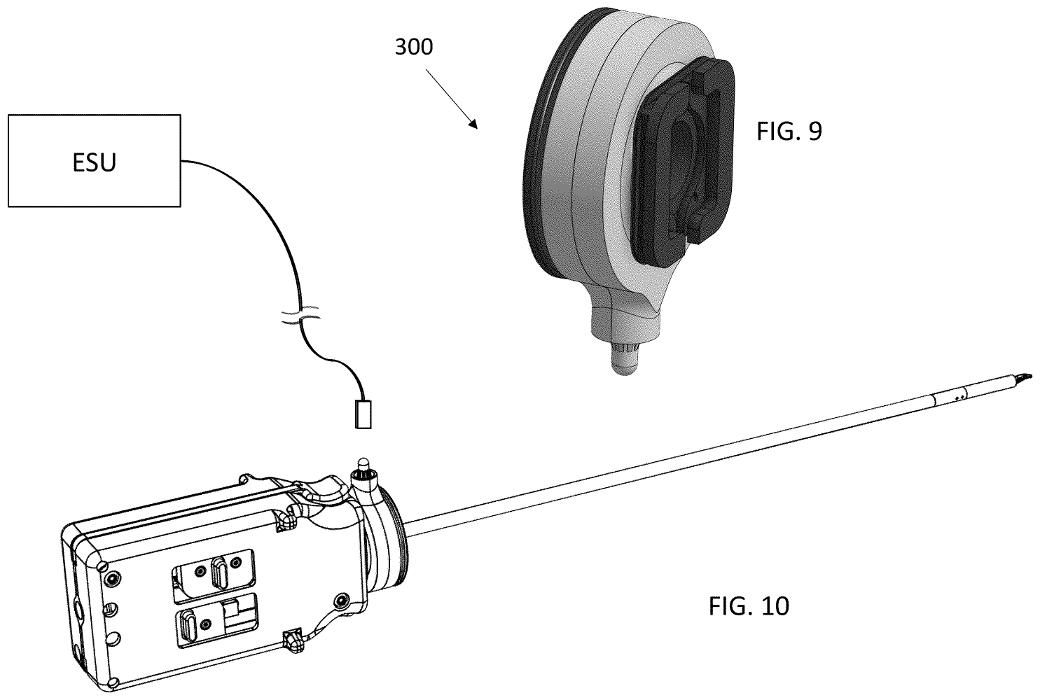

A slip ring assembly 200 for a monopolar instrument is shown in FIGS. 8 and 9. This configuration is similar to the bipolar configuration, with a flange 201, slip ring, 302, brush 312, pin 314 and base 304. It differs primarily in that only a single slip ring, brush, and wire are used. Therefore the seat for the brush is disposed within two housing sections 310a, 310b that sandwich the brush between them and come together to form the connector 318.

In preferred embodiments, the bipolar and monopolar housing sections are designed to fit on a common base and flange with different ring configurations. Both the monopolar and bipolar slip ring assemblies were created to be modular and have identical interfaces with the instrument adapter 110.

While certain embodiments have been described above, it should be understood that these embodiments are presented by way of example, and not limitation. It will be apparent to persons skilled in the relevant art that various changes in form and detail may be made therein without departing from the scope of the invention characterized by the claims. This is especially true in light of technology and terms within the relevant art(s) that may be later developed. Moreover, features of the various disclosed embodiments may be combined in various ways to produce various additional embodiments.

All patents, patent applications and printed publications referred to above, including for purposes of priority, are incorporated herein by reference.

We claim:

1. An electrosurgical instrument for robotic surgical procedures, the instrument comprising:
an elongate shaft having an effector, the elongate shaft having a longitudinal axis extending from a proximal end to a distal end of the elongate shaft;
at least one electrical conductor extending through the shaft for conducting electrical current to the end effector;
a slip ring assembly at a proximal end of the elongate shaft, the slip ring assembly comprising
a connector configured to electrically connect to a cable of an electrosurgical unit, the connector including at least one conductive pin;
a non-conductive housing;
a conductive spring member disposed within the housing and electrically coupled to the connector; and
a conductive ring disposed within the housing and positioned in electrical contact with the conductive spring member
wherein the non-conductive housing and spring member are axially rotatable about the longitudinal axis relative to the elongate shaft and conductive ring.

2. The electrosurgical instrument of claim 1, further including an adapter mounted to the proximal end of the elongate shaft the adapter configured to be mounted to a robotic manipulator, and wherein the slip ring assembly is mounted to the adapter such that the non-conductive housing and spring member are axially rotatable about the longitudinal axis relative to the elongate shaft, adapter, and conductive ring.

3. A method of using an electrosurgical instrument in a robotic surgical procedure, comprising:
providing a surgical instrument comprising
an elongate shaft having an effector, the elongate shaft having a longitudinal axis extending from a proximal end to a distal end of the elongate shaft;
at least one electrical conductor extending through the shaft for conducting electrical current to the end effector; and
an adapter mounted to the proximal end of the elongate shaft, the adapter including a connector;
mounting the adapter to a robotic manipulator arm;
connecting a cable of an electrosurgical unit to the connector, the connector having an orientation relative to the electrosurgical unit;
positioning the distal end of the instrument in contact with tissue of a patient and operating the electrosurgical unit to conduct current through the connector, wire and distal end to tissue of the patient; and
operating the robotic manipulator arm to axially rotate the adapter and elongate shaft relative to the patient, wherein during axial rotation of adapter and elongate shaft, the connector maintains the orientation.

4. The method of claim 3, wherein the adapter further includes a slip ring assembly comprising the connector including at least one conductive pin, a non-conductive housing, a conductive spring member disposed within the housing and electrically coupled to the connector, and a conductive ring disposed within the housing and positioned in electrical contact with the conductive spring member, wherein during the operating step the elongate shaft, adapter and conductive ring are axially rotatable about the longitudinal axis relative to non-conductive housing and spring member.
